(19) United States
(12) Patent Application Publication
(10) Pub. No.: US 2026/0272266 A1
(43) Pub. Date: Sep. 17, 2026

(54) VISION-BASED SCENE SEGMENTATION AND ACTION CLASSIFICATION

(71) Applicant: Auris Health, Inc., Santa Clara, CA (US)

(72) Inventor: Saif Iftekar Sayed, Foster City, CA (US)

(73) Assignee: Auris Health, Inc., Santa Clara, CA (US)

(21) Appl. No.: 19/564,612

(22) Filed: Mar. 12, 2026

Related U.S. Application Data
(60) Provisional application No. 63/772,207, filed on Mar. 14, 2025.

(51) Int. Cl. A61B 1/00; A61B 1/018; A61B 1/05; A61B 18/00; A61B 18/26; G06V 10/12; G06V 10/44; G06V 10/764; G06V 10/77; G06V 10/774; G06V 10/82; G06V 20/40; G16H 30/40

(52) U.S. Cl. CPC A61B 1/000096; A61B 1/018; A61B 1/05; A61B 18/26; G06V 10/12; G06V 10/44; G06V 10/764; G06V 10/7715; G06V 10/774; G06V 10/82; G06V 20/41; G06V 20/46; G16H 30/40; A61B 2018/00511; A61B 2018/00982; G06V 2201/031; G06V 2201/034

(57) ABSTRACT

This disclosure provides methods, devices, and systems for classifying video frames. The present implementations more specifically relate to vision-based scene segmentation and action classification. In some aspects, a machine learning model may be trained to infer, from images or video captured by a camera disposed on the distal end of a medical instrument (such as a scope), information about a working channel instrument and/or other objects in a field-of-view (FOV) of the camera. In some implementations, a first machine learning model may be trained to extract features associated with an object (such as a working channel instrument, an anatomy, or a target within the anatomy) from one or more frames of video depicting the object, and a second machine learning model may be trained to infer an action associated with the object, for each frame of the one or more frames, based on the features extracted from the frame.

### CROSS-REFERENCE TO RELATED APPLICATIONS

This application claims priority and benefit under 35 U.S.C. § 119(e) to U.S. Provisional Patent Application No. 63/772,207, filed Mar. 14, 2025, which is incorporated herein by reference in its entirety.

### TECHNICAL FIELD

This disclosure relates generally to medical systems, and specifically to vision-based scene segmentation and action classification.

### DESCRIPTION OF RELATED ART

Many medical procedures, such as bronchoscopy, laparoscopy, ureteroscopy, or percutaneous nephrolithotomy (PCNL), involve a series of complex steps that require careful movement and positioning of medical tools or instruments inside a patient's body. For example, to remove urinary stones from the bladder and ureter, a user (such as a physician or technician) can insert a ureteroscope into the urinary tract through the urethra. A ureteroscope includes a camera at its distal end configured to enable visualization of the urinary tract. The user drives the ureteroscope to a target anatomical site and uses a medical instrument (such as a catheter or basket device) to extract the urinary stone. In some instances, the user may insert another medical instrument (such as a laser) into the target anatomical site to break up the stone into smaller fragments that can be more easily extracted. The success or failure of such medical procedures often depends on various factors, including the user's skill, the patient's anatomy, and the quality of any tools or equipment used to perform the procedure.

Some medical systems can record or document case logs related to a medical procedure, which can be analyzed for training purposes and/or medical research. Case logs can include any runtime data that a medical system generates or collects during a medical procedure and/or data input by a user. Example case log data may include recordings of images or video captured by the camera of the ureteroscope and/or telemetry from medical instruments or other devices used in the procedure. However, raw case log data may not be suitable for evaluating the success of the medical procedure or the user's performance. Further, a medical system collecting case log data may not have access to telemetry from some instruments used in the procedure (such as third-party instruments that are not integrated with the medical system). Thus, there is a need to provide greater insights about a medical procedure using existing case log data available to the medical system.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method of classifying frames of video. The method includes steps of obtaining video captured by a camera disposed on a distal end of a medical instrument; detecting a presence of an object in a series of frames of the video; extracting features associated with the object from one or more frames in the series of frames using a first machine learning model; and inferring, for each frame of the one or more frames, an action associated with the object using the features extracted from the frame and a second machine learning model.

Another innovative aspect of the subject matter of this disclosure can be implemented in a method of training a video classifier. The method includes steps of obtaining a plurality of videos depicting an object; training a first machine learning model to extract features associated with the object based on the plurality of videos; extracting a plurality of features from the plurality of videos using the trained first machine learning model; and training a second machine learning model to classify actions associated with the object in individual frames of video based on the plurality of features extracted by the first machine learning model.

Another innovative aspect of the subject matter of this disclosure can be implemented in a controller for a medical system, including a processing system and a memory. The memory stores instructions that, when executed by the processing system, cause the controller to obtain video captured by a camera disposed on a distal end of a medical instrument; detect a presence of an object in a series of frames of the video; extract features associated with the object from one or more frames in the series of frames using a first machine learning model; and infer, for each frame of the one or more frames, an action associated with the object using the features extracted from the frame and a second machine learning model.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows example medical system components, according to some implementations.

FIG. 3 shows an example positioning of instruments within an anatomy, according to some implementations.

FIG. 4 shows a block diagram of an example system for annotating video of a medical procedure, according to some implementations.

FIG. 5 shows a block diagram of an example action classification system, according to some implementations.

FIG. 6 shows a block diagram of an example machine learning system, according to some implementations.

FIG. 7 shows a block diagram of an example controller for a medical system, according to some implementations.

FIG. 8 shows an illustrative flowchart depicting an example operation for classifying frames of video, according to some implementations.

FIG. 9 shows an illustrative flowchart depicting an example operation for training a video classifier, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures involve a series of complex steps that require careful movement and positioning of medical tools or instruments inside a patient's body. Some medical procedures can now be performed, at least in part, by a robotic system or apparatus, which can aid the physician in navigating or positioning such medical instruments. For example, a physician can control a robotic system to advance and navigate a medical instrument (such as a scope) within an anatomy. The robotic system may include, or may be coupled to, one or more display devices that can provide information to assist the physician in navigating the medical instrument. Example suitable information may include real-time images captured by the medical instrument, a visualization to indicate a position and/or orientation (also referred to as a “pose”) of the medical instrument, and/or guidance regarding the procedure, among other examples. Such information can be captured or obtained using various sensors and/or cameras disposed on or otherwise coupled to the medical instrument.

Some medical procedures involve the use of shaft-type instruments, such as endoscopes, which may be inserted into an anatomy and advanced to a target anatomical site. Some endoscopes having a working channel or lumen through which another instrument (also referred to as a “working channel instrument”) can be inserted. An example suitable working channel instrument is a laser fiber that can be used for laser lithotripsy or otherwise breaking down objects within the anatomy (such as kidney stones). Telemetry and/or information about the working channel instrument can provide valuable insights about various aspects or phases of a medical procedure (such as the duration of laser usage, inadvertent tissue interactions, the precise locations of laser application, and the effectiveness of various laser settings on different stone compositions). However, some working channel instruments may not be communicatively coupled to a medical system. As a result, the medical system may be unable to acquire telemetry from the working channel instruments.

Some shaft-type instruments, such as endoscopes, have cameras disposed on their distal ends. The camera captures real-time images that can be used for navigating the distal tip within an anatomy (also referred to as “endoscopic vision”). Aspects of the present disclosure recognize that such images can also be used to infer information about the working channel instrument and/or other instruments or objects in the field-of-view (FOV) of the camera. Example suitable inferences can include a presence (or absence) of an instrument, whether the instrument is idle or performing an action (such as lasing a target), and whether the action is performed on the target or the surrounding anatomy, among other examples. In some aspects, a machine learning model may be trained to infer such information from images or video captured by a scope. In some implementations, a first machine learning model may be trained to extract features associated with an object from one or more frames of video depicting the object, and a second machine learning model may be trained to infer an action associated with the object, for each frame of the one or more frames, based on the features extracted from the frame.

Although certain aspects of the present disclosure are described in detail herein in the context of renal, urological, or nephrological procedures, such as kidney stone removal and treatment procedures, it should be understood that such context is provided for convenience and clarity, and the concepts disclosed herein are applicable to any suitable medical procedure. Description of the renal or urinary anatomy and associated medical issues and procedures is presented herein to aid in the description of the concepts disclosed herein. However, it should be understood that these techniques and systems can be implemented in the context of any medical procedure involving visualization inside an anatomy (such as using an endoscope).

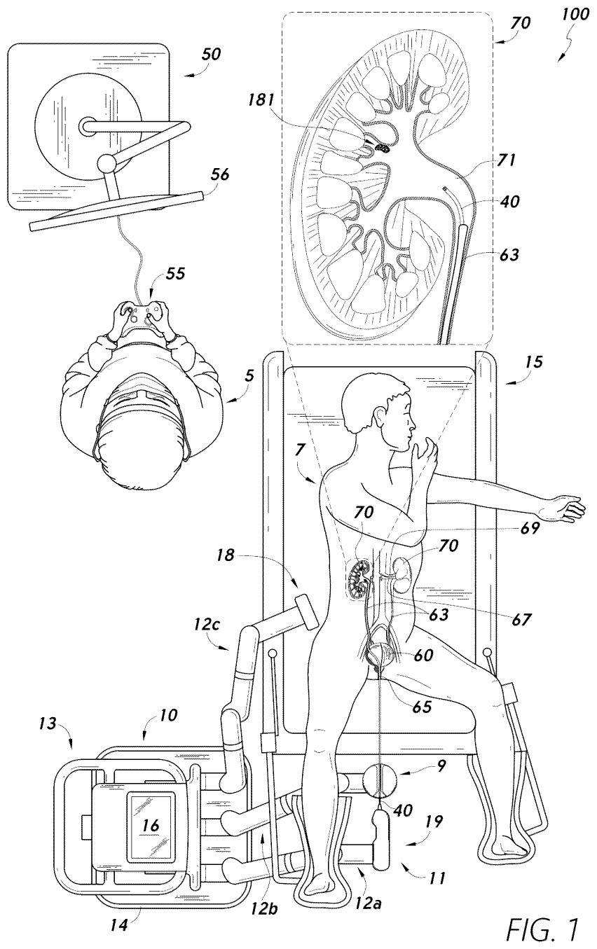

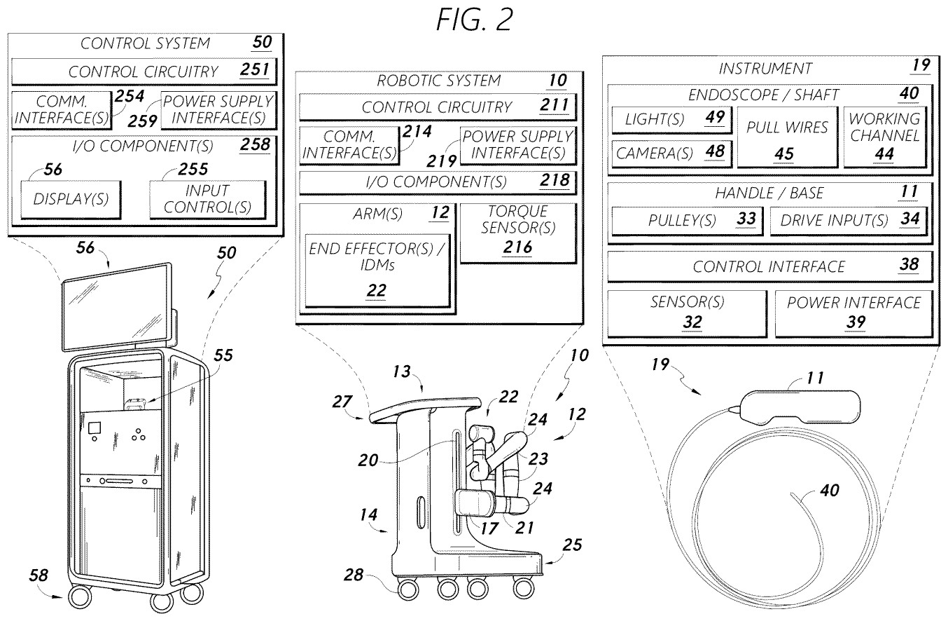

FIG. 1 shows an example medical system 100, according to some implementations. FIG. 2 shows detailed example implementations of certain components of the medical system 100 shown in FIG. 1. The description below of the medical system 100 and various components (e.g., robotic components) thereof may be understood with reference to either or both of FIGS. 1 and 2. Generally, robotic solutions can provide relatively higher precision, superior control, and/or superior hand-eye coordination with respect to control of certain instruments compared to strictly manual solutions.

The medical system 100 includes a robotic system 10 configured to engage with and/or control an instrument 19 (e.g., endoscope/ureteroscope) including a proximal base 11 (e.g., handle) and a shaft 40 coupled to the base 11 at a proximal portion thereof. The robotic system 10 can be configured to facilitate execution of a medical procedure and can be arranged in a variety of positions and configurations, depending on the procedure. The medical system 100 can include a table 15 configured to hold the subject patient 7.

The robotic system 10 can include one or more robotic arms 12 configured to engage with and/or control the instrument 19 to perform one or more aspects of a procedure. As shown, each robotic arm 12 can include multiple arm segments 23 coupled to joints 24, which can provide multiple degrees of movement/freedom. The robotic system 10 can have an instrument feeder instrument 9 coupled to an arm 12b thereof to facilitate robotic advancement of the instrument 19. Another arm 12a may have the instrument base 11 coupled thereto.

The robotic system 10 can be electrically and/or communicatively coupled to any component of the medical system 100, such as to a control system 50, the table 15, an electromagnetic (EM) field generator 18, and/or the instrument 19. Robotic system 10 also includes a power supply interface 219, which may receive power to drive robotic system 10 via wire, battery, and/or any other suitable kind of power source. In addition, robotic system 10 one example includes various input/output (I/O) components 218 configured to assist the operator 5 or others in performing a medical procedure.

In some examples, robotic system 10 may be communicatively coupled with control system 50 via communication interfaces 214, 254. For example, the robotic system 10 may be configured to receive control signals from the control system 50 to perform certain operations, such as to position one or more of the robotic arms 12, manipulate (e.g., advance, articulate) the instrument 19, and so on. In response, the robotic system 10 can control, using certain control circuitry 211, actuators 217, and/or other components of the robotic system 10, to perform the operations. For example, the control circuitry 211 may control articulation of the shaft of the instrument 19 by actuating drive output(s) of the end effector 22 coupled to the instrument base 11. In some implementations, the robotic system 10 and/or control system 50 is/are configured to receive images and/or image data from the instrument 19 representing internal anatomy of the patient 7.

The robotic system 10 can include a support structure 14 (also referred to as a “column”), a robotic system base 25, and a console 13 at the top of the column 14. The console 13 also includes a handle 27 to assist with maneuvering and stabilizing the robotic system 10. The column 14 may include one or more arm supports 17 (also referred to as a “carriage”) for supporting the deployment of the one or more robotic arms 12 (three shown in FIG. 1). The robotic arms 12 of the present example generally comprise robotic arm bases 21 and end effectors 22, separated by a series of linking arm segments 23 that are connected by a series of joints 24, each joint comprising one or more independent actuators. Each actuator 217 may comprise an independently controllable motor. The arm support 17 may be configured to vertically translate along the column 14. In some implementations, the arm support 17 can be connected to the column 14 through slots 20 that guide the vertical translation of the arm support 17. The robotic system base 25 can include wheel-shaped casters 28.

The console 13 can provide both a user interface for receiving user input and a display 16 (e.g., screen or a dual-purpose device such as, for example, a touchscreen) to provide the physician/user 5 with both pre-operative and intra-operative data. Potential pre-operative data on the console/display 16 or display 56 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse.

Articulation of the shaft 40 of the instrument 19 may be controlled robotically, such as through operation of a robotic manipulator of the robotic system 10. The combination of the end effector 22 and the instrument base 11, as well as any intervening mechanics or couplings (e.g., sterile adapter), can be referred to as a manipulator assembly. The term “end effector” is used herein according to its broad and ordinary meaning and may refer to any type of robotic manipulator device, component, and/or assembly. In implementations in which an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector.

Furthermore, the terms “manipulator,” “robotic manipulator,” and “robotic manipulator assembly” are used according to their broad and ordinary meanings, and may refer to a robotic end effector and/or sterile adapter or other adapter component coupled to the end effector, either collectively or individually. For example, the terms “robotic manipulator” and “robotic manipulator assembly” may refer to one or more drive outputs, rails, arms, pulleys, gears, couplings, belts, guides, or the like, whether embodied in a robotic end effector, sterile adapter, and/or other component(s). Robotic manipulators of the present disclosure can include connectors to transfer pneumatic pressure, electrical power, electrical signals, and/or optical signals between the robotic system 10 and a coupled instrument, such as the instrument 19.

The control system 50 can be configured to interface with the robotic system 10, provide information regarding the procedure, and/or perform a variety of other operations. In some examples, the control system 50 can include wheel-shaped casters 58. The control system 50 includes a power supply interface 259, which may receive power to drive control system 50 via wire, battery, and/or any other suitable kind of power source. A control circuitry 251 of the control system 50 may provide signal processing and execute control algorithms to achieve the functionality of the medical system 100 as described herein.

The control system 50 can include various input/output (I/O) components 258 configured to assist the physician 5 or others in performing a medical procedure. For example, the I/O components 258 can be configured to allow for user input to control/navigate the instrument 19 and/or other robotically controlled instrument within the patient 7. In some examples, the physician 5 can provide input to the control system 50 and/or robotic system 10 via one or more input controls 255, wherein in response to such input, control signals can be sent to the robotic system 10 to manipulate the instrument 19. The control system 50 can receive real-time images that are captured by the instrument 19 and display the real-time images via the display(s) 56. Additionally, or alternatively, the control system 50 can receive signals (e.g., analog, digital, electrical, acoustic/sonic, pneumatic, tactile, hydraulic, etc.) from a medical monitor and/or a sensor associated with the patient 7, and the display(s) 56 can present information regarding the health or environment of the patient 7.

The system 100 can include certain control circuitry configured to perform certain of the functionality described herein, including the control circuitry 211 of the robotic system 10 and the control circuitry 251 of the control system 50. That is, the control circuitry of the system 100 may be part of the robotic system 10, the control system 50, or some combination thereof. Therefore, any reference herein to control circuitry may refer to circuitry embodied in a robotic system, a control system, or any other component of a medical system. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions.

Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in implementations in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211,251 may be entirely locally maintained/disposed or may be remotely located at least in part (e.g., communicatively coupled indirectly via a local area network and/or a wide area network).

The control circuitry 211 and/or control circuitry 251 may be communicatively coupled to one or more torque sensors 216 configured to generate signals indicative of torque on one or more actuators of the robotic system 10, such as articulation pulley drive gears/outputs. The torque sensor(s) 216 may have any suitable or desirable configuration. For example, the torque sensor(s) 216 can act as a sensed mounting structure or load cell. In some implementations, the torque sensor(s) 216 is/are configured as a reactive torque sensor that measures torque induced strain using one or more self-contained strain gauges to create a load cell. Although torque sensors 216 of a robotic system are described herein in the context of determining force on pull wire pulleys of a steerable instrument, such references may be understood to represent any type of sensor(s) or sensing mechanism configured to generate signals indicative of forces on pull wires and/or associated pulleys/actuators, such as strain gauges or the like. References herein to torque sensors or other types of force sensors (e.g., strain gauges) can be any type of sensor configured to measure force/load on a robotic actuator, whether such force is rotational or linear in nature. That is, although rotational robotic output drives are disclosed in some contexts herein, it should be understood that inventive concepts disclosed herein apply to other types of actuators, such as linear drives.

As noted above, robotic system 10 may be communicatively coupled with control system 50 via communication interfaces 214, 254. The various components of the system 100 can be communicatively coupled to each other over a network, which can include a wireless network and/or a wired network. Example networks include one or more personal area networks (PANs), local area networks (LANs), wide area networks (WANs), Internet area networks (IANs), cellular networks, the Internet, personal area networks (PANs), body area network (BANs), etc. Furthermore, in some implementations, the various components of the systems can be connected for data communication, fluid exchange, power exchange, and so on via one or more support cables, tubes, or the like.

The control system 50 and/or the robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to the respective control circuitry. In some implementations, the user may engage the user controls 55 to command robotic shaft articulation, as described herein.

The instrument 19 (e.g., endoscope) includes a base 11 (e.g., handle) coupled to an elongate shaft 40. The shaft 40 of the instrument 19 can include one or more lights 49 and one or more cameras or other imaging devices 48. The shaft 40 can further include one or more working channels 44, which may run a length of the shaft 40. The instrument 19 can be powered through a power interface 39 and/or controlled through a control interface 38, each or both of which may interface with a robotic end effector of the robotic system 10. The instrument 19 may further comprise one or more sensors 32, such as pressure sensors and/or other force-reading sensors, which may be configured to generate signals indicating position and/or forces experienced at/by one or more components of the instrument 19.

The instrument 19 includes certain mechanisms for causing the shaft 40 to articulate/deflect with respect to an axis thereof. For example, the instrument 19 may include one or more drive inputs 34 associated with one or more pulleys 33 that are configured to tension/untension pull wires 45 to cause articulation of the shaft 40.

In an example use case, if the patient 7 has a kidney stone (or stone fragment) 181 located in a kidney 70, the physician 5 may perform a procedure to remove the stone 181 through the urinary tract (63, 60, 65). In some implementations, the physician 5 can interact with the control system 50 and/or the robotic system 10 to cause/control the robotic system 10 to advance and navigate the instrument 40 (e.g., a ureteroscope) from the urethra 65, through the bladder 60, up the ureter 63, and into the renal pelvis 71 and/or calyx network of the kidney 70 where the stone 181 is located. The physician 5 can further interact with the control system 50 and/or the robotic system 10 to cause/control the advancement of a basketing device or other instrument (such as a laser fiber) through a working channel of the instrument 40 to facilitate capture and removal of a kidney stone or stone fragment.

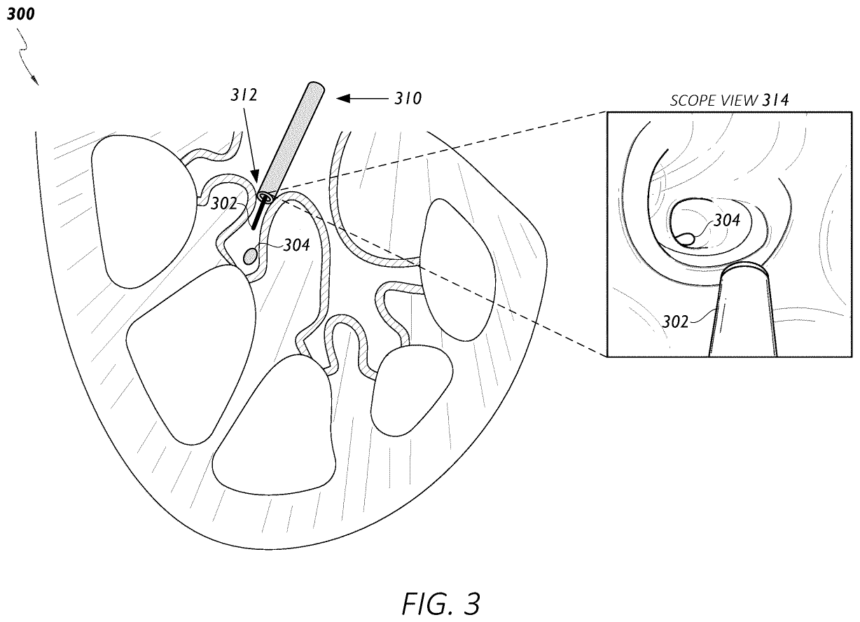

FIG. 3 shows an example positioning of instruments 310 and 302 within an anatomy 300, according to some implementations. In the example of FIG. 3, the anatomy 300 is depicted as a kidney, the instrument 310 is a scope having a camera 312 disposed on its distal tip (such as an endoscope or ureteroscope), and the instrument 302 (also referred to herein as a “working channel instrument”) is inserted through a working channel of the scope 310. In some implementations, the working channel instrument 302 may be a laser fiber that can be used to break down a kidney stone 304 into smaller fragments (such as via laser lithotripsy). However, various other working channel instruments can be used when performing other medical procedures and/or other phases of the same procedure.

As shown in FIG. 3, the distal tip of the working channel instrument 302 extends beyond the distal opening of the scope 310 as it is brought into contact (or proximity) with the stone 304. As a result, the working channel instrument 302 can be seen in images or video 314 (also referred to as a “scope view”) captured by the camera 312. The scope view 314 depicts a field-of-view (FOV) of the camera 312. In some aspects, the scope view 314 may be updated in real-time (or near real-time) to provide live visualization inside the anatomy 300. For example, the scope 310 and/or camera 312 may be communicatively coupled to a medical system (such as the medical system 100 of FIG. 1) that can display the scope view 314 to a user (such as on the display 56 of FIGS. 1 and 2). Thus, the scope view 314 can help the user navigate the working channel instrument 302 to the stone 304 and monitor a lasing and/or extraction of the stone 304.

In some implementations, the medical system may record or otherwise store the images and/or video displayed via the scope view 314. For example, the recording can be stored as case log data associated with the medical procedure. As used herein, the term “case log data” refers to any runtime data that a medical system generates or collects during a medical procedure and/or data input by a user. In some implementations, the case log data may include telemetry from one or more components of the medical system (such as the scope 310). Information about the working channel instrument 302 can provide valuable insights about various aspects or phases of the procedure (such as the duration of laser usage, inadvertent tissue interactions, the precise locations of laser application, and the effectiveness of various laser settings on different stone compositions). However, in some instances, the working channel instrument 302 may not be communicatively coupled to the medical system. As a result, the medical system may be unable to acquire telemetry (or case log data) from the working channel instrument 302.

Aspects of the present disclosure recognize that the scope view 314 can be used to infer information about the working channel instrument 302 and/or other instruments or objects in the FOV of the camera 312. Example suitable inferences can include a presence (or absence) of an instrument, whether the instrument is idle or performing an action (such as lasing a target), and whether the action is performed on the stone 304 or the surrounding anatomy 300, among other examples. In some implementations, a machine learning model may be trained to infer such information from images or video captured by a scope. Machine learning is a technique for improving the ability of a computer system to perform a certain task. Machine learning generally comprises a training phase and an inferencing phase. During the training phase, a machine learning system is provided with a large volume of raw training data. The machine learning system analyzes the training data to learn a set of rules (also referred to as the “machine learning model”) that can be used to describe various patterns associated with the data. During the inferencing phase, the machine learning system can infer patterns from new data using the learned set of rules.

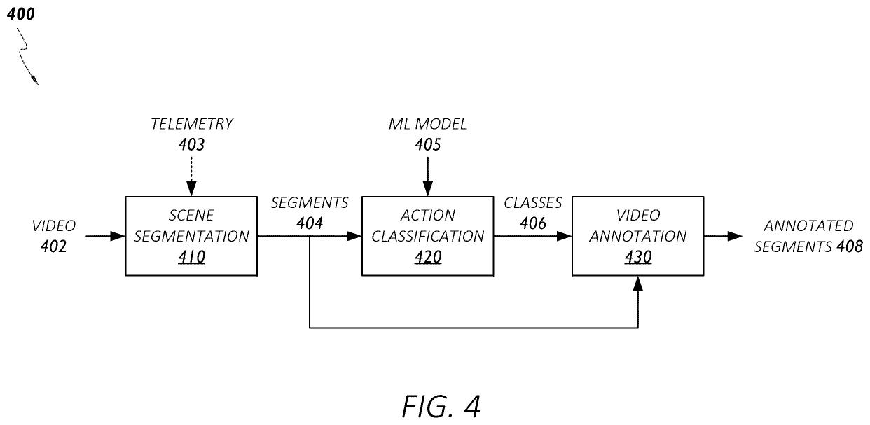

FIG. 4 shows a block diagram of an example system 400 for annotating video of a medical procedure, according to some implementations. The system 400 can be implemented as part of a control system for a medical system (such as the medical system 100 of FIG. 1). With reference for example to FIG. 2, the system 400 may be implemented by the control circuitry 251 and/or the control circuitry 211.

The system 400 is configured to receive video 402 (or images) captured by a camera disposed on the distal end of an endoscope (such as the endoscope 40 of FIGS. 1 and 2 or the scope 310 of FIG. 3). The video 402 depicts an FOV of the endoscope at any given time (such as the scope view 314 of FIG. 3), which can help a user navigate the endoscope and/or a working channel instrument within an anatomy and/or luminal network. For example, the video 402 may represent a stream or sequence of images or frames that are continuously captured (in real-time) by the camera. As such, the video 402 can depict any movements and/or actions of instruments and/or other objects in the FOV of the endoscope (such as the working channel instrument 302, the stone 304, or other features of the anatomy 300 of FIG. 3). In some implementations, the system 400 may use the video 402 to classify one or more actions performed by an instrument (such as a laser) in the FOV of the endoscope and annotate one or more frames of the video 402 with the corresponding actions.

The system 400 includes a scene segmentation component 410, an action classification component 420, and a video annotation component 430. The scene segmentation component 410 is configured to partition the video 402 into one or more segments 404 based on a presence (or absence) of an object for tracking (such as the instrument) in each segment 404. In some implementations, the scene segmentation component 410 may detect the presence of the object in the video 402 using computer vision techniques (such as object detection). For example, the scene segmentation component 410 can analyze the video 402 using an object detection model that is trained or otherwise configured to detect objects of a particular type (such as a laser fiber) based on known features associated with the object (such as textures and/or geometry).

In some other implementations, the scene segmentation component 410 may detect the presence of the object based on telemetry 403 from one or more components of the medical system coupled to or otherwise associated with the object (such as one or more robotic arms that hold or manipulate the instrument). For example, the scene segmentation component 410 can determine or estimate the position of the instrument relative to a position of the scope by comparing a pose of the robotic arm coupled to the instrument with a pose of the robotic arm coupled to the scope. The scene segmentation component 410 can match the timestamps associated with the robotic arm poses to corresponding segments of the video 402.

The scene segmentation component 410 combines or aggregates the video frames based on the presence (or absence) of the object so that each resulting segment 404 of the video 402 includes a series of contiguous video frames in which the object is detected. In some implementations, the scene segmentation component 410 may output only the segments 404 of video 402 that contain the object. In some other implementations, the scene segmentation component 410 also may output segments of the video 402 that do not contain the object. In such implementations, the scene segmentation component 410 may label each segment to indicate the presence or absence of the object and/or refrain from providing any segments to the action classification component 420 that do not contain the object.

The action classification component 420 is configured to classify one or more actions associated with the object in each of the segments 404. In some aspects, the action classification component 420 may distinguish between active and idle states of the object in each segment 404. For example, where the object is a laser, the active state can correspond to a lasing operation (such as where the laser emits a concentrated beam of light) and the idle state can correspond to an absence of light being emitted by the laser. As another example, where the object is a stone or anatomical feature, the active state can correspond to movement or displacement of the stone or anatomical feature (such as when the stone is broken down into smaller fragments or displaced via irrigation, aspiration, and/or respiration) and the idle state can correspond to an absence of movement by the stone or anatomical feature. In some implementations, the action classification component 420 may first determine whether the object is active or idle in each frame of the segments 404 and further classify the type of action associated with the object in the frames for which the object is active. More specifically, the action classification component 420 may output one or more classes 406 for each of the segments 404 indicating the action(s) of the object in one or more frames of the segment 404. Where the object is a laser, example suitable classes 406 can include lasing_stone (where the laser beam is directed at a stone) and lasing_tissue (where the laser beam is directed at the anatomy or tissue surrounding the stone).

In some aspects, the action classification component 420 may determine the classes 406 using one or more machine learning (ML) models 405. In some implementations, the ML models 405 may be trained to perform action segmentation on the segments 404. In some other implementations, the ML models 405 may be trained to perform action detection on the segments 404. Action segmentation is a technique for subdividing a continuous video stream or segment into snippets of even shorter length, where each snippet is associated with a particular action or activity. In other words, an action segmentation model assigns a particular activity to each frame of video and attempts to determine when each activity ends and the next activity begins. Where the object is a laser, example suitable activities can include lasing_stone, lasing_tissue, and lasing_idle (where the laser does not emit a beam). By contrast, action detection is a technique for identifying specific actions within a video stream or segment while treating the rest of the video as background (BG). For example, rather than classifying snippets of lasing_idle activity, an action detection model may ignore such activity as BG.

The video annotation component 430 is configured to annotate each segment 404 with the classes 406 to produce annotated segments 408. For example, the annotated segments 408 may include labels or tags identifying the frames and/or times for which the actions and/or activities associated with the classes 406 are detected. In some implementations, the annotated segments 408 may be stored by the medical system as case log data that can be reviewed and/or analyzed postoperatively. For example, after performing a laser lithotripsy procedure, a user (such as a physician or technician) can review the annotated segments 408 to evaluate the lasing strategy alongside laser placement, laser settings, and/or stone properties, while identifying any outliers, to look for ways to improve the performance of the procedure (such as in regards to instrument placement, fluidics performance, or any other factors within the user's control). The annotated segments 408 can also be used to predict the duration of a future laser lithotripsy procedure (such as based on stone volume, location, and lasing times from past procedures).

In some other implementations, the medical system may analyze the annotated segments 408 intraoperatively to provide real-time feedback to the user. For example, the medical system may determine, based on the annotated segments 408 associated with a laser lithotripsy procedure, that the current placement of the laser is historically associated with longer procedure times or greater risk of lasing the surrounding anatomy. In such instances, the medical system may instruct or otherwise guide the user to move the laser to an improved location (such as closer to a kidney stone).

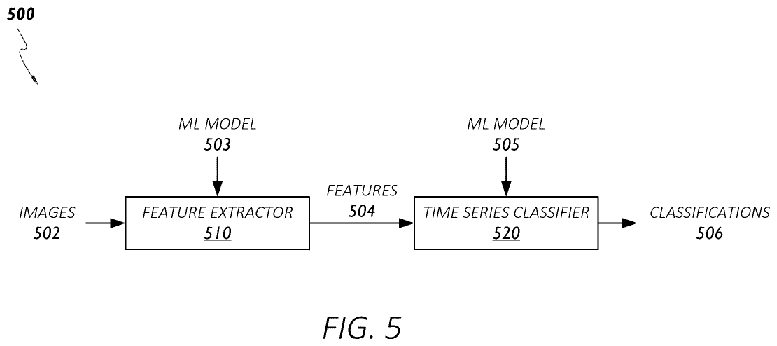

FIG. 5 shows a block diagram of an example action classification system 500, according to some implementations. In some implementations, the action classification system 500 may be one example of the action classification component 420 of FIG. 4. More specifically, the action classification system 500 is configured to determine one or more classifications 506 for a sequence of images 502. With reference to FIG. 4, each sequence of images 502 may be one example of a segment 404 of the video 402, and the classifications 506 may be one example of the classes 406. In other words, each of the images 502 may depict an object (such as an instrument, object, or anatomical feature) in the FOV of an endoscope and each of the classifications 506 may indicate an action and/or activity of the object depicted by one of the images 502 (such as a lasing activity or idleness of the laser).

The action classification system 500 includes a feature extractor 510 and a time series classifier 520. The feature extractor 510 is configured to extract a respective set of features 504 from each of the images 502. In other words, the feature extractor 510 performs frame-level feature extraction, for example, by analyzing each image 502 to capture relevant data about objects, scenes, textures, motion, and/or other visual elements within the image 502. In some implementations, the feature extractor 510 may extract the features 504 using an ML model 503. These features 504 can include generic frame-level features that capture the context of the scene (rather than individual segmentation masks or object detections). Example suitable machine learning architectures include convolutional neural networks (CNNs), vision transformers (ViTs), and multimodal learning architectures that combine text and vision inputs (such as video snippets), among other examples.

In some implementations, the ML model 503 may be trained to implement action segmentation. In such implementations, the ML model 503 may be trained to extract features associated with actions or activities of the object that can be assigned to every image 502 in the series. Where the object is a laser, example suitable actions and activities may include lasing_idle, lasing_stone, lasing_tissue, and lasing_active (which can be associated with any type of lasing action), among other examples. In some other implementations, the ML model 503 may be trained to implement action detection. In such implementations, the ML model 503 may be trained to extract features associated with a smaller subset of object actions. Where the object is a laser, example suitable actions may include lasing_active, lasing_idle, and lasing_stone, among other examples (where lasing_idle can be ignored).

The time series classifier 520 is configured to track the features 504 across multiple images 502 (or frames of video) and classify each image 502 based on the temporal relationships between the features 504. In other words, the time series classifier 520 may analyze the relationships and dependencies between sequential frames to represent the dynamic aspects of the video. More specifically, the time series classifier 440 may determine a respective classification 506 for each image 502 indicating the type of action or activity depicted by the image 502 based on the features 504 extracted from the image 502 as well as features extracted from one or more images preceding and/or following the image 502. In some implementations, the time series classifier 520 may determine the classifications 506 using an ML model 505. Example suitable machine learning architectures include temporal convolutional networks (TCNs), long short-term memory (LSTM) networks, and transformers, among other examples.

In some implementations, the ML model 505 may be trained to implement action segmentation. In such implementations, the ML model 505 may be trained to classify each action or activity of the object depicted by the images 502 (such as lasing_active, lasing_idle, lasing_stone, or lasing_tissue). In other words, the classification 506 for each image 502 indicates a particular action or activity. In some other implementations, the ML model 505 may be trained to implement action detection. In such implementations, the ML model 505 may be trained to classify only a subset of object actions depicted by the images 502 (such as lasing_active, lasing_stone, or lasing_tissue). In other words, the classification 506 for each image 502 can either indicate a particular action or background activity (such as BG).

In some aspects, the ML models 503 and 505 may be trained on the same dataset but in multiple stages. For example, the ML model 503 (also referred to as a “feature extraction model”) can be trained in a first stage and the ML model 505 (also referred to as a “temporal tracking model”) can be trained in a second stage using the outputs of the ML model 503 trained in the first stage. In other words, the weights of the feature extraction model 503 can be frozen or “locked in” after the first stage to avoid variations or discrepancies in the features used to train the temporal tracking model 505 in the second stage.

In some aspects, the feature extraction model 503 may be trained using a CNN-based architecture. A CNN is a particular type of artificial neural network that processes data in a manner similar to the human visual system. As such, CNNs are well-suited for computer vision applications. For example, each neuron in a CNN responds to a respective subset of the data from a previous layer, located within its receptive field, by outputting an “activation” representing a higher level abstraction of a feature in its receptive field. The receptive fields of the neurons in a given layer are combined to cover the entire input from the previous layer (similar to how the receptive fields of cortical neurons in the brain cover the entire visual field). The set of activations output by each layer is collectively referred to as a “feature map.”

Thus, a CNN can be trained to classify input images using ground truth labels (such as lasing_active, lasing_idle, lasing_stone, and/or lasing_tissue) so that the CNN learns a set of features associated with each label or classification. The CNN can be trained using cross-entropy loss to ensure that the feature extraction model 503 learns distinctive features. The CNN can also be trained using contrastive loss to maximize the distance between features associated with different classes while minimizing the distance between features associated with the same classes. When the feature extraction model 503 is deployed, the feature map having the highest level of abstraction may be taken as the output of the CNN (such as the feature map provided as input to a classification layer). In other words, the output of the model 503 may correspond to the input (rather than the output) of the classification layer of the CNN.

In some other aspects, the feature extraction model 503 may be trained using a ViT architecture. A ViT applies the transformer model (designed for natural language processing) to computer vision tasks. For example, a ViT subdivides an image into one or more patches having a fixed size (similar to tokenization of a text string in natural language processing), then flattens or serializes each patch into a vector and maps the vector to an embedding. An “embedding” is a numerical vector, in any high-dimensional space, having a magnitude and direction that represents a real-world object or set of objects. Some ViTs implement an encoder-decoder architecture that can be trained to recreate, at its output, the same image received at its input (similar to autoencoders). As a result of encoding and decoding an input image, the ViT is forced to learn the latent features of the image.

Thus, a ViT can be trained to learn the latent features of images known to depict object activity (such as lasing_active, lasing_idle, lasing_stone, and/or lasing_tissue), even without any annotations or ground truth labels. For example, the encoder for a ViT can be trained to convert each image into one or more vector embeddings representing latent features of the image, and the decoder for the ViT can be trained to reconstruct the original image from the vector embeddings output by the encoder. The ViT can be trained using an image reconstruction loss to ensure that the output of the decoder closely matches the input to the encoder (such as within a threshold range of error). When the feature extraction model 503 is deployed, the vector embeddings may be taken as the output of the ViT. In other words, the output of the model 503 may correspond to the output of the encoder (rather than the output of the decoder).

Still further, in some aspects, the feature extraction model 503 may be trained using a multi-modal learning architecture. For example, some transformer-based architectures can combine multiple encoders, including a text encoder trained on textual content (such as labels or annotations) and a video encoder trained on images or video (such as a ViT). This multi-modal transformer architecture creates a joint embedding space for video sequences and text-based descriptions of actions associated with the video sequences. By sharing a joint embedding space, the text encoder and the video encoder can be trained to align more closely with one another by comparing the video embeddings to the text embeddings using a predefined similarity metric (such as cosine similarity). The multi-modal model can be trained to maximize the similarity between matching pairs of video and text embeddings while minimizing the similarity for non-matching pairs of video and text embeddings.

Thus, a multi-modal encoder can learn to match snippets of video depicting object actions and/or activity with corresponding text descriptions of the actions or activity (such as “lasing active,” “lasing idle,” “lasing stone,” and/or “lasing tissue”). In some implementations, the labels may be re-encoded to provide greater context or description of the activity. For example, video snippets classified as lasing_stone can be reclassified into more granular labels, such as lasing_stone_pointing_center (where the laser is pointing to the center of a stone), lasing_stone_pointing_boundary (where the laser is pointing to the edge of a stone), lasing_stone_while_high_fluid_flow (where the lasing conditions include high irrigation), lasing_stone_while_high_fragment_flow (where the lasing conditions include a high flow of stone fragments), green_laser_pointing_stone (where the laser emits a green colored beam), lasing_stone_after_k_seconds (where the lasing is activated after a given duration), or lasing_stone_before_lasing_tissue (where the laser beam moves from the stone to the tissue), among other examples. Such relabeling of the actions can help the text encoder to learn enhanced contextual information, which can help the video encoder to extract more spatial context from each video snippet, resulting in a more robust feature extraction model 503.

The trained feature extraction model 503 is then used to generate features (such as vector embeddings) for training the temporal tracking model 505. In some implementations, the temporal tracking model 505 may be trained using a TCN architecture. TCNs are specialized neural network architectures designed for sequence modeling and time series analysis. More specifically, TCNs use causal convolutions that look at past data inputs to predict future values. Thus, a TCN can be trained to classify each frame of a video based on object action or activity detected in the frame and temporal dependencies associated with such actions or activity in a sequence of video frames. The TCN can be trained using cross-entropy loss to ensure that the temporal tracking model 505 learns distinctive actions and/or activity. The TCN can also be trained using a smoothing loss to improve the consistency of classifications for neighboring or adjacent video frames (such as to ensure that neighboring classifications are the same).

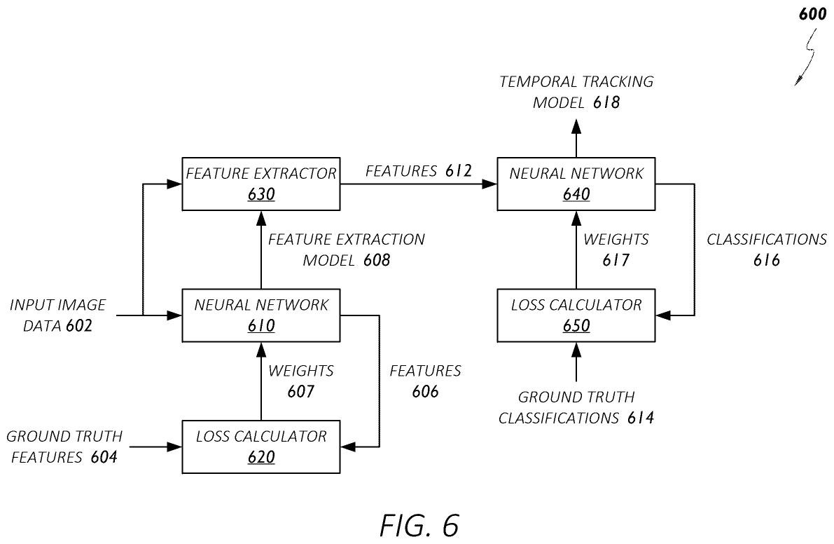

FIG. 6 shows a block diagram of an example machine learning system 600, according to some implementations. The machine learning system 600 is configured to produce neural network models, including a feature extraction model 608 and a temporal tracking model 618, based at least in part on input image data 602 representing a large volume of images captured by cameras disposed on medical instruments (such as endoscopes). With reference for example to FIGS. 1 and 2, the control system 50 may capture or acquire the input image data 602 via the endoscope 40 over one or more medical procedures.

The machine learning system 600 includes a first neural network 610, a first loss calculator 620, a feature extractor 630, a second neural network 640, and a second loss calculator 650. In some aspects, the machine learning system 600 may train the neural networks 610 and 640 in multiple stages. Deep learning is a particular form of machine learning in which the inferencing and training phases are performed over multiple layers. Deep learning architectures are often referred to as “artificial neural networks” due to the manner in which information is processed (similar to a biological nervous system). For example, each layer of an artificial neural network may be composed of one or more “neurons.” Each layer of neurons may perform a different transformation on the output data from a preceding layer so that the final output of the neural network results in the desired inferences. The set of transformations associated with the various layers of the network is referred to as a “neural network model.” Example suitable neural networks include CNNs, ViTs, TCNs, LSTMs, recurrent neural networks (RNNs), and transformers, among other examples.

In a first training stage, the machine learning system 600 trains the first neural network 610 to reproduce ground truth features 604 based on the input image data 602. The ground truth features 604 represent a desired output of the first neural network 610 for a given set of input image data 602. Thus, in some implementations, the ground truth features 604 may represent contextual information about each image or frame of video, including any information associated with actions or activities of an object depicted by the image. The first neural network 610 receives the input image data 602 and attempts to extract the ground truth features 604 from the input image data 602. For example, the first neural network 610 may form a network of connections across multiple layers of artificial neurons that begin with the input image data 602 and lead to a set of features 606 at its output. The connections are weighted to result in a set of features 606 that closely resembles the ground truth features 604.

The first training stage is performed over multiple iterations. In each iteration, the first neural network 610 produces a respective set of features 606 based on weighted connections across the layers of artificial neurons, and the first loss calculator 620 updates the weights 607 associated with the connections based on an amount of loss (or error) between the output features 606 and the ground truth features 604. The first neural network 610 may output the weighted connections as the feature extraction model 608 when certain convergence criteria are met (such as when the loss falls below a threshold level or a predetermined number of training iterations have been performed). In some implementations, the first neural network 610 may implement a CNN and the features 606 may include a feature map. In some other implementations, the first neural network 610 may implement a ViT and the features 606 may include vector embeddings. Still further, in some implementations, the first neural network 610 may implement a multi-modal architecture including a text encoder and a video encoder, and the features 606 may include text embeddings and/or video embeddings.

The feature extractor 630 is configured to extract a set of features 612 from the input image data 602 using the trained feature extraction model 608. In some implementations, the feature extractor 630 may be one example of the feature extractor 510 of FIG. 5 and the feature extraction model 608 may be one example of the ML model 603 of FIG. 5. In other words, the feature extractor 630 implements the trained feature extraction model 608 to extract frame-level features from the input image data 602. These features 612 can then be used to train the temporal tracking model 618. In this way, the weights 607 of the feature extraction model 608 are frozen or locked in after the first stage of training to avoid variations or discrepancies in the features used to train the temporal tracking model 618.

In a second training stage, the machine learning system 600 trains the second neural network 640 to reproduce ground truth classifications 614 based on the features 612 extracted from the input image data 602. The ground truth classifications 614 represent a desired output of the second neural network 640 for a given set of features 612. Thus, in some implementations, the ground truth classifications 614 may include actions or activities associated with objects depicted by each image or frame of video. In some implementations, the ground truth classifications 614 may indicate whether the object is active or idle. The second neural network 640 receives the features 612 and attempts to infer the ground truth classifications 614 from the features 612. For example, the second neural network 640 may form a network of connections across multiple layers of artificial neurons that begin with a set of features 612 and lead to a respective classification 616 at its output. The connections are weighted to result in a classification 616 that closely resembles the ground truth classification 614.

The second training stage is performed over multiple iterations. In each iteration, the second neural network 640 produces a respective classification 616 based on weighted connections across the layers of artificial neurons, and the second loss calculator 650 updates the weights 617 associated with the connections based on an amount of loss (or error) between the output classifications 616 and the ground truth classifications 614. The second neural network 640 may output the weighted connections as the temporal tracking model 618 when certain convergence criteria are met (such as when the loss falls below a threshold level or a predetermined number of training iterations have been performed). In some implementations, the second neural network 640 may implement a TCN.

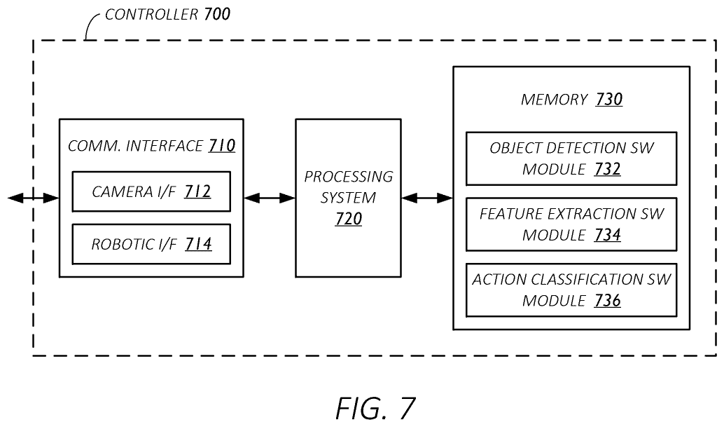

FIG. 7 shows a block diagram of an example controller 700 for a medical system, according to some implementations. In some implementations, the controller 700 may be one example of any of the systems 400 or 500 of FIGS. 4 and 5, respectively, or any of the control circuitry 251 or 211 of FIG. 2. More specifically, the controller 700 is configured to classify frames of video captured by a camera disposed on a medical instrument.

The controller 700 includes a communication interface 710, a processing system 720, and a memory 730. The communication interface 710 is configured to communicate with one or more components of a medical system. More specifically, the communication interface 710 includes a camera interface (I/F) 712 for communicating with a camera disposed on a distal end of a medical instrument (such as the camera 48 of FIG. 2) and a robotic interface (I/F) 714 for communicating with a robotic system (such as the robotic system 10 of FIGS. 1 and 2). In some implementations, the camera interface 712 may obtain video captured by the camera disposed on the distal end of the medical instrument.

The memory 730 may include a non-transitory computer-readable medium (including one or more nonvolatile memory elements, such as EPROM, EEPROM, Flash memory, or a hard drive, among other examples) that may store the following software (SW) modules: an object detection SW module 732 to detect a presence of an object in a series of frames of the video; a feature extraction SW module 734 to extract features associated with the object from one or more frames in the series of frames using a first machine learning model; and an action classification SW module 736 to infer an action associated with the object, for each frame of the one or more frames, based on using the features extracted from the frame and a second machine learning model.

The processing system 720 may include any suitable one or more processors capable of executing scripts or instructions of one or more software programs stored in the controller 700 (such as in the memory 730). For example, the processing system 720 may execute the object detection SW module 732 to detect a presence of an object in a series of frames of the video. The processing system 720 also may execute the feature extraction SW module 734 to extract features associated with the object from one or more frames in the series of frames using a first machine learning model. Further, the processing system 720 may execute the action classification SW module 736 to infer an action associated with the object, for each frame of the one or more frames, based on using the features extracted from the frame and a second machine learning model.

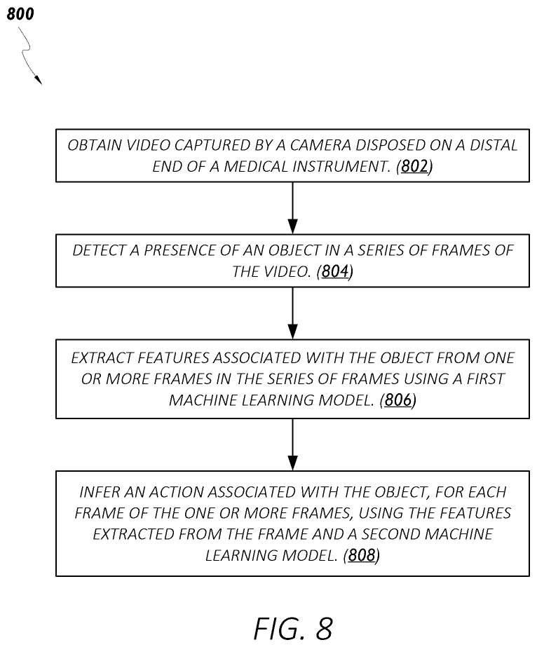

FIG. 8 shows an illustrative flowchart depicting an example operation 800 for classifying frames of video, according to some implementations. In some implementations, the example operation 800 may be performed by the system 400 of FIG. 4.

The system obtains video captured by a camera disposed on a distal end of a medical instrument (802). The system detects a presence of an object in a series of frames of the video (804). In some implementations, the system may further receive telemetry from a robotic system configured to manipulate the medical instrument, where the presence of the object is detected based at least in part on the received telemetry. The system extracts features associated with the object from one or more frames in the series of frames using a first machine learning model (806). In some implementations, the features extracted from each frame may include contextual information about the frame. The system infers an action associated with the object, for each frame of the one or more frames, based on using the features extracted from the frame and a second machine learning model (808). In some implementations, the system may further annotate the video with the action inferred for each frame of the one or more frames.

In some implementations, the action may indicate whether the object is in an active state or an idle state. In some implementations, the medical instrument may be configured to be inserted into an anatomy, and the object may be a target within the anatomy. In some other implementations, the medical instrument may be a scope having a working channel, and the object may be a working channel instrument configured to be inserted through the working channel of the scope. In some implementations, the working channel instrument may be a laser. In some implementations, the action may include lasing an anatomy or lasing a target within the anatomy.

In some implementations, the first machine learning model may include a CNN and the one or more features may include a feature map output by the CNN. In some other implementations, the first machine learning model may include a ViT and the one or more features may include vector embeddings output by the ViT. Still further, in some implementations, the first machine learning model may include a multi-modal architecture including a text encoder and a video encoder, and the one or more features may include at least a text embedding output by the text encoder or a video embedding output by the video encoder. In some implementations, the second machine learning model may include a TCN.

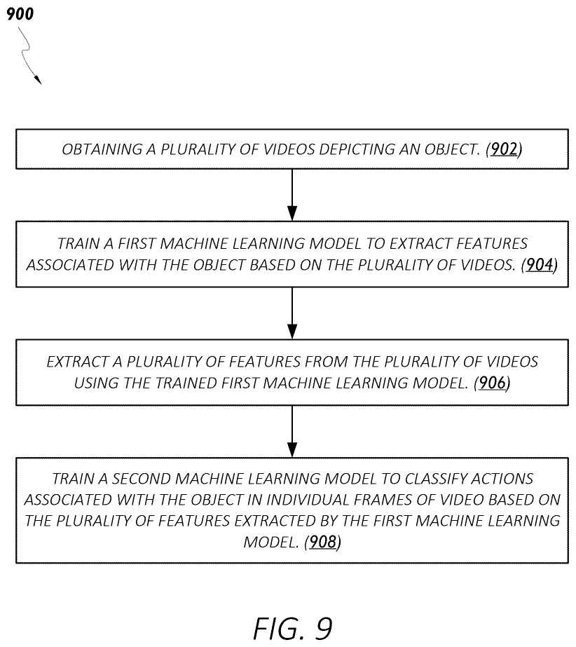

FIG. 9 shows an illustrative flowchart depicting an example operation 900 for training a video classifier, according to some implementations. In some implementations, the example operation may be performed by the system 500 of FIG. 5.

The system obtains a plurality of videos depicting an object (902). The system trains a first machine learning model to extract features associated with the object based on the plurality of videos (904). The system extracts a plurality of features from the plurality of videos using the trained first machine learning model (906). The system trains a second machine learning model to classify actions associated with the object in individual frames of video based on the plurality of features extracted by the first machine learning model (906).

In some implementations, the first machine learning model may include a CNN and the plurality of features may include a feature map output by the CNN. In some other implementations, the first machine learning model may include a ViT and the plurality of features may include vector embeddings output by the ViT. Still further, in some implementations, the first machine learning model may include a multi-modal architecture including a text encoder and a video encoder, and the plurality of features may include at least a text embedding output by the text encoder or a video embedding output by the video encoder. In some implementations, the second machine learning model may include a TCN.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method of classifying frames of video, comprising:
obtaining video captured by a camera disposed on a distal end of a medical instrument;
detecting a presence of an object in a series of frames of the video;
extracting features associated with the object from one or more frames in the series of frames using a first machine learning model; and
inferring an action associated with the object, for each frame of the one or more frames, using the features extracted from the frame and a second machine learning model.

2. The method of claim 1, further comprising:
receiving telemetry from a robotic system configured to manipulate the medical instrument, the presence of the object detected based at least in part on the received telemetry.

3. The method of claim 1, wherein the action indicates whether the object is in an active state or an idle state.

4. The method of claim 1, wherein the medical instrument is configured to be inserted into an anatomy, and the object comprises a target within the anatomy.

5. The method of claim 1, wherein the medical instrument comprises a scope having a working channel, and the object comprises a working channel instrument configured to be inserted through the working channel of the scope.

6. The method of claim 5, wherein the working channel instrument is a laser.

7. The method of claim 6, wherein the action includes lasing an anatomy or lasing a target within the anatomy.

8. The method of claim 1, wherein the first machine learning model comprises a convolutional neural network (CNN) and the one or more features include a feature map output by the CNN.

9. The method of claim 1, wherein the first machine learning model comprises a vision transformer (ViT) and the one or more features include vector embeddings output by the ViT.

10. The method of claim 1, wherein the first machine learning model comprises a multi-modal architecture including a text encoder and a video encoder, and wherein the one or more features include at least a text embedding output by the text encoder or a video embedding output by the video encoder.

11. The method of claim 1, wherein the second machine learning model comprises a temporal convolutional network (TCN).

12. The method of claim 1, wherein the features extracted from each frame include contextual information about the frame.

13. The method of claim 1, further comprising:
annotating the video with the action inferred for each frame of the one or more frames.

14. A method of training a video classifier, comprising:
obtaining a plurality of videos depicting an object;
training a first machine learning model to extract features associated with the object based on the plurality of videos;
extracting a plurality of features from the plurality of videos using the trained first machine learning model; and
training a second machine learning model to classify actions associated with the object in individual frames of video based on the plurality of features extracted by the first machine learning model.

15. The method of claim 14, wherein the first machine learning model comprises a convolutional neural network (CNN) and the plurality of features includes a feature map output by the CNN.

16. The method of claim 14, wherein the first machine learning model comprises a vision transformer (ViT) and the plurality of features includes vector embeddings output by the ViT.

17. The method of claim 14, wherein the first machine learning model comprises a multi-modal architecture including a text encoder and a video encoder, and wherein the plurality of features include a text embedding output by the text encoder or a video embedding output by the video encoder.

18. The method of claim 14, wherein the second machine learning model comprises a temporal convolutional network (TCN).

19. The method of claim 14, wherein the features include contextual information about individual frames of video.

20. A controller for a medical system, comprising:
a processing system;
a memory storing instructions that, when executed by the processing system, cause the controller to:
obtain video captured by a camera disposed on a distal end of a medical instrument;
detect a presence of an object in a series of frames of the video;
extract features associated with the object from one or more frames in the series of frames using a first machine learning model; and
infer an action associated with the object, for each frame of the one or more frames, using the features extracted from the frame and a second machine learning model.
